(19) United States
(12) Patent Application Publication
(10) Pub. No.: US 2024/0351928 A1
(43) Pub. Date: Oct. 24, 2024

(54) SYSTEMS AND METHOD OF BIODEGRADATION OF RECALCITRANT COMPOUNDS USING FUNGAL REACTORS

(71) Applicant: THE TRUSTEES OF COLUMBIA UNIVERSITY IN THE CITY OF NEW YORK, New York, NY (US)

(72) Inventors: Julian John Alexander VAN DER MADE, New York, NY (US); Kartik CHANDRAN, New York, NY (US); Elizabetth Amy LANDIS, Newburgh, NY (US); Ruby LAI, Seattle, WA (US)

(73) Assignee: THE TRUSTEES OF COLUMBIA UNIVERSITY IN THE CITY OF NEW YORK, New York, NY (US)

(21) Appl. No.: 18/637,984

(22) Filed: Apr. 17, 2024

Related U.S. Application Data
(60) Provisional application No. 63/460,174, filed on Apr. 18, 2023.

(51) Int. Cl. C02F 3/34; C02F 1/467; C02F 1/72; C02F 101/34; C02F 101/36; C02F 103/34; C12M 1/00; C12M 1/06; C12M 1/12; C12M 1/42; C12N 1/14

(52) U.S. Cl. CPC C02F 3/347; C02F 1/4672; C02F 1/722; C12M 25/02; C12M 27/06; C12M 29/06; C12M 35/02; C12N 1/14; C02F 2101/34; C02F 2101/36; C02F 2103/34; C02F 2305/026

(57) ABSTRACT

A fungal culture is provided that incorporates concentrations of Fenton reactants and white rot fungi biomass. These components synergize to degrade recalcitrant organic compounds such as lignins, perfluorochemicals including PFOA, plastics, and related compounds, particularly with 1.5 mM H₂O₂, 1 mM Fe²⁺, and Phanerochaete chrysosporium exhibiting lignin degrading capabilities comparable to cultures of Fenton-only reaction mediums with significantly higher concentrations of hydrogen peroxide. Both the fungi and the Fenton reactants work to degrade the organic compounds. Additionally, the Fenton reactants impose oxidative stress on fast-growing microbial competitors such as E. coli, selectively inhibiting the competing bacteria and their disadvantageous effects on white rot fungi activity. The white rot fungi recycle Fe(II) ions to reduce Fenton reactant input burden. An electro-Fenton reaction process can further reduce this burden by replenishing H₂O₂. The resulting systems and methods demonstrate more economical and sustainable treatment of recalcitrant organic compounds.

### CROSS-REFERENCE TO RELATED APPLICATIONS

This application claims the benefit of U.S. Provisional Patent Application No. 63/460,174, filed Apr. 18, 2023, which is incorporated by reference as if disclosed herein in its entirety.

### BACKGROUND

Recalcitrant organic compounds present a significant obstacle to the breakdown and recycling of waste materials via bacterial and/or fungal digestion. These organic compounds are present in industrial or commercial effluent streams and waste byproducts which are harmful to human health and to the environment. Further, these organic compounds are known to persist in the environment for extended periods of time due to limited degradability by conventional means. By way of example, in natural environments, lignin encases cellulosic and hemicellulosic materials, making them unavailable for anaerobic digestion and reducing the efficacy of bacterial digestion of cellulosic wastes.

There are certain fungi that are capable of degrading lignin, e.g., through enzymatic modification. In particular, white-rot fungi such as P. Chrysosporium express extremely active lignolytic enzymes including lignin peroxidase (LiP) and manganese peroxidase (MnP). However, efforts to incorporate organic compound-degrading fungi into systems and methods for the digestion of waste materials have been hindered by the co-proliferation of bacteria, which typically out-compete the fungi in culture and hamper the fungal digestive bioprocesses.

What is desired, therefore, are systems and methods that harness the digestive capabilities of fungi to continuously degrade a range of organic waste materials on an industrial scale.

### SUMMARY

Aspects of the present disclosure are directed to fungal culture including a plurality of organic compound (OC)-degrading fungi, a concentration of Fe(II) ions, and a concentration of hydrogen peroxide. In some embodiments, the OC-degrading fungi includes white rot fungi. In some embodiments, the white rot fungi includes Phanerochaete chrysosporium. In some embodiments, the concentration of Fc (II) ions is between about 0.1 mM and about 1 mM. In some embodiments, the concentration of hydrogen peroxide is less than about 20 mM. In some embodiments, the concentration of hydrogen peroxide is between about 0.5 mM and about 5 mM. In some embodiments, the concentration of hydrogen peroxide is about 1.5 mM. In some embodiments, the concentration of Fe(II) ions is about 1 mM. In some embodiments, the fungal culture includes a concentration of organic compounds including wherein the organic compounds include lignin, perfluorochemicals, plastics, or combinations thereof.

Aspects of the present disclosure are directed to a system for degrading organic compounds including a bioreactor including one or more reservoirs, a fungal culture in the one or more reservoirs, and a feedstream in fluid communication with the reservoir. In some embodiments, the feedstream includes a concentration of one or more organic compounds. In some embodiments, the fungal culture includes a plurality of white rot fungi, a concentration of Fe(II) ions, and a concentration of hydrogen peroxide. In some embodiments, the system includes one or more pairs of electrodes in electrical communication with the fungal culture. In some embodiments, the organic compounds include lignin, perfluorochemicals, plastics, or combinations thereof. In some embodiments, the white rot fungi includes Phanerochaete chrysosporium. In some embodiments, the concentration of hydrogen peroxide is between about 0.5 mM and about 5 mM. In some embodiments, the concentration of hydrogen peroxide is about 1.5 mM and the concentration of Fe(II) ions is about 1 mM.

In some embodiments, the system includes an incubation apparatus positioned within the bioreactor. In some embodiments, the incubation apparatus includes a shaft extending longitudinally along the reservoir, one or more substrates positioned on and extending from the shaft, and a motor configured to rotate the shaft. In some embodiments, the substrate is a disk extending at least substantially orthogonally from the shaft.

Aspects of the present disclosure are directed to a method of degrading organic compounds including preparing a fungal culture including a plurality of white rot fungi, a concentration of Fe(II) ions, and hydrogen peroxide, and adding an amount of one or more organic compounds to the fungal culture. In some embodiments, the concentration of Fe(II) ions in the composition is about 1 mM and the concentration of hydrogen peroxide in the composition is about 1.5 mM. In some embodiments, the white rot fungi includes Phanerochaete chrysosporium. In some embodiments, the organic compounds include lignin, perfluorochemicals, plastics, or combinations thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIGS. 1A-1B are graphs portraying lignin degradation data for fungal cultures according to embodiments of the present disclosure and Fenton reactants;

FIG. 2 is a graph portraying the effects on Fenton reactants of fungal cultures according to embodiments of the present disclosure;

FIG. 3 is a flowchart of a process for lignin degradation according to embodiments of the present disclosure;

FIG. 4 is a graph portraying the effects on competitive bacterial growth of fungal cultures according to embodiments of the present disclosure;

FIG. 5A is a graph portraying the effects of Fenton reactant concentration on biomass;

FIG. 5B is a graph portraying lignin degradation data for fungal cultures according to embodiments of the present disclosure;

FIG. 6 is a schematic representation of a system for degrading organic compounds according to embodiments of the present disclosure; and

FIG. 7 is a chart of a method of degrading organic compounds according to embodiments of the present disclosure.

### DETAILED DESCRIPTION

Some embodiments of the present disclosure are directed to a fungal culture for degrading organic compounds. In some embodiments, the organic compounds include lignin, perfluorochemicals, plastics, or combinations thereof. The organic compounds can be from any suitable source thereof, e.g., industrial effluents, synthetic waste materials, naturally-occurring materials, etc., or combinations thereof. In some embodiments, the fungal culture includes a plurality of organic compound (OC)-degrading fungi. In some embodiments, the fungal culture includes two or more different strains of OC-degrading fungi. In some embodiments, the composition of the OC-degrading fungi component of the fungal culture is non-static, e.g., evolves over time, exhibits enhanced fungal diversity over time, etc. In some embodiments, the OC-degrading fungi includes white rot fungi. In some embodiments, the white rot fungi includes Phanerochaete chrysosporium.

In some embodiments, the fungal culture includes reactants for facilitating Fenton reaction chemistry in the culture, hereinafter also referred to as the “Fenton reactants.” In some embodiments, the Fenton reactants include Fe(II) ions. In some embodiments, the Fe(II) ions are generated via dissociation of one or more iron compounds in the fungal culture. In some embodiments, the concentration of Fe(II) ions in the fungal culture is between about 0.1 mM and about 1 mM. In some embodiments, the concentration of Fe(II) ions in the fungal culture is about 1 mM.

In some embodiments, the Fenton reactants include a concentration of hydrogen peroxide (H₂O₂). In some embodiments, the concentration of hydrogen peroxide is less than about 20 mM. In some embodiments, the concentration of hydrogen peroxide is between about 0.5 mM and about 5 mM. In some embodiments, the concentration of hydrogen peroxide is about 1.5 mM. In some embodiments, the concentration of Fe(II) ions is about 1 mM and the concentration of hydrogen peroxide is about 1.5 mM.

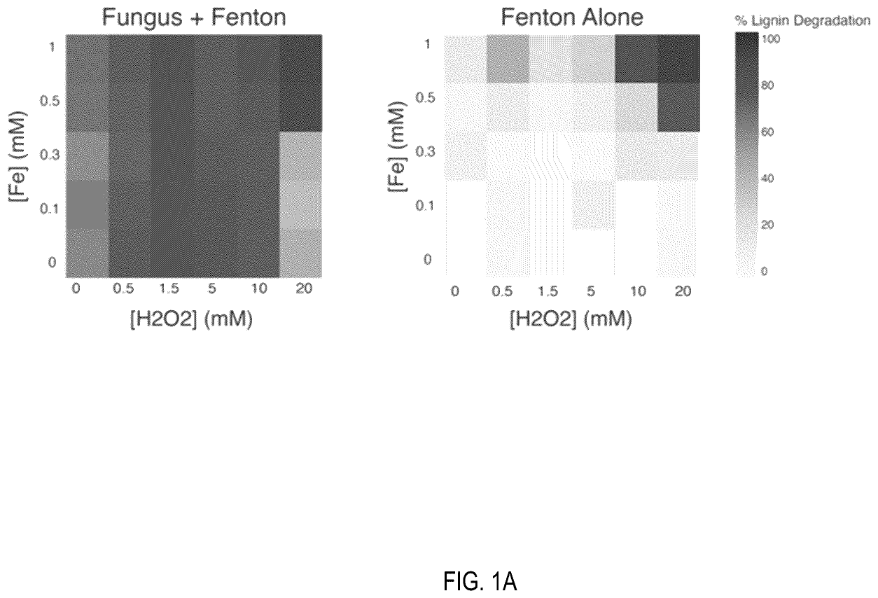

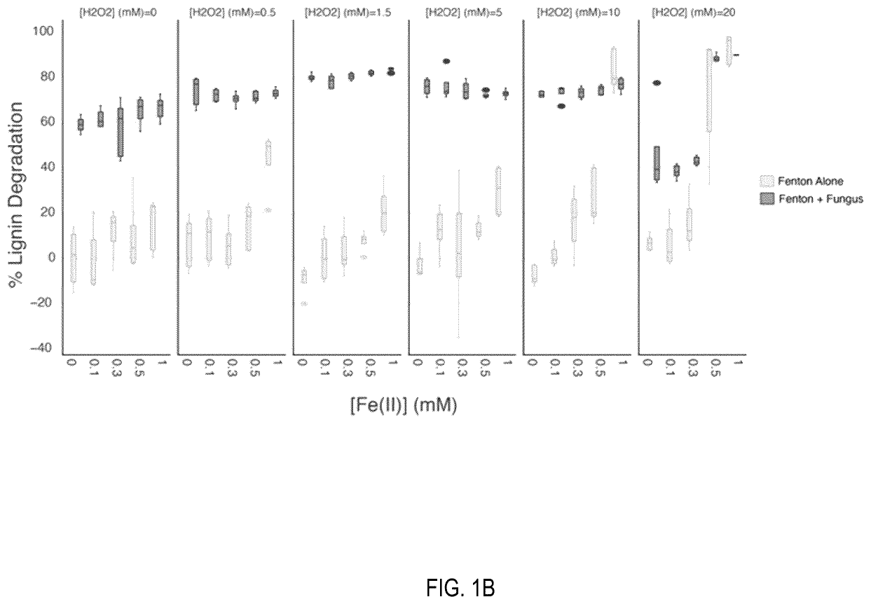

Referring now to FIG. 1, the fungal cultures of the present disclosure work to degrade OC targets, including recalcitrant OCs. The activity of the Fenton reactants generates reaction products including a concentration of hydroxyl radicals, hydroxide ions, and Fe(III) ions. The hydroxyl radials help in the degradation of OCs, such as through conversion of non-degradable lignin to degradable lignin. Concurrently, the OC-degrading fungi express their own OC degradation constructs in the form of OC-degrading enzymes, e.g., lignin peroxidase (LiP), manganese peroxidase (MnP), etc., or combinations thereof. Referring to FIG. 2, OC-degrading fungi can also replenish concentrations of Fe(II) ions, lowering the Fenton reactant input burden on the fungal culture.

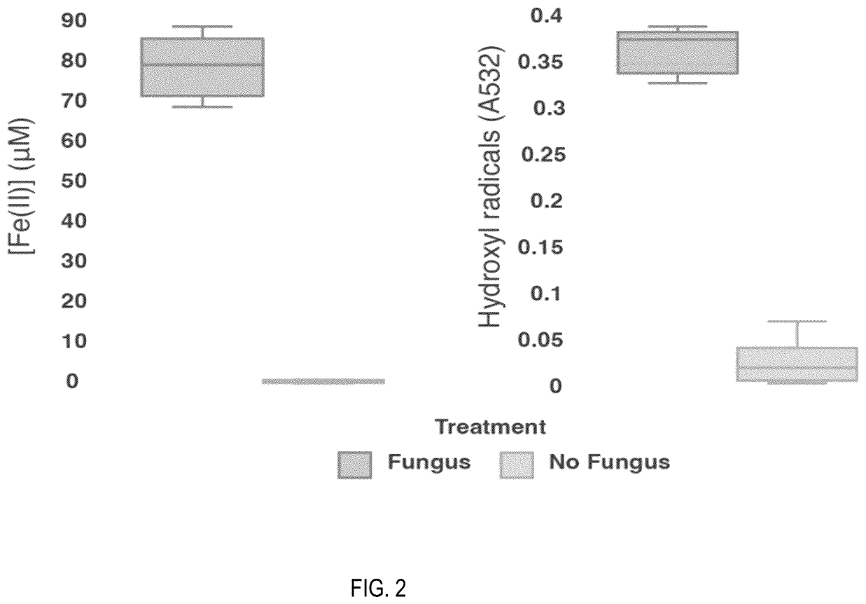

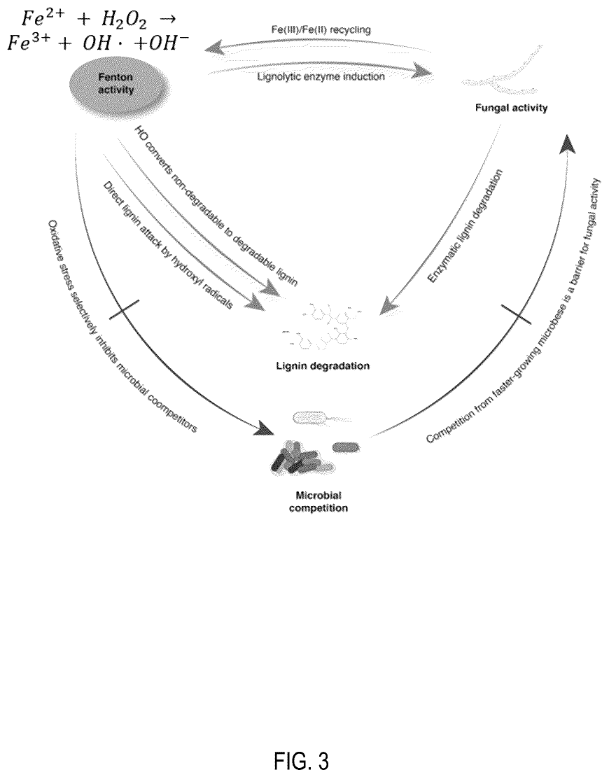

Referring now to FIG. 3, a flowchart is provided to visualize OC degradation by fungal cultures consistent with embodiments of the present disclosure. As discussed above, the Fenton reactants generate reaction products such as hydroxyl radicals, hydroxide ions, and Fc (III) ions. While the hydroxyl radicals work themselves to degrade OCs, the reaction products further exert exhibit oxidative stress on bacteria competing with the OC-degrading fungi in the fungal culture. This stress greatly inhibits proliferation of the bacteria (see FIG. 4), reducing overall competition that can be a barrier to fungal growth and limit fungal enzymatic OC degradation. The additional fungal biomass is also able to recycle Fe(III) ions back to Fe(II) to stimulate OC degradation via Fenton chemistry. Thus, the components of the fungal cultures consistent with the embodiments of the present disclosure act synergistically to enhance degradation of recalcitrant OCs.

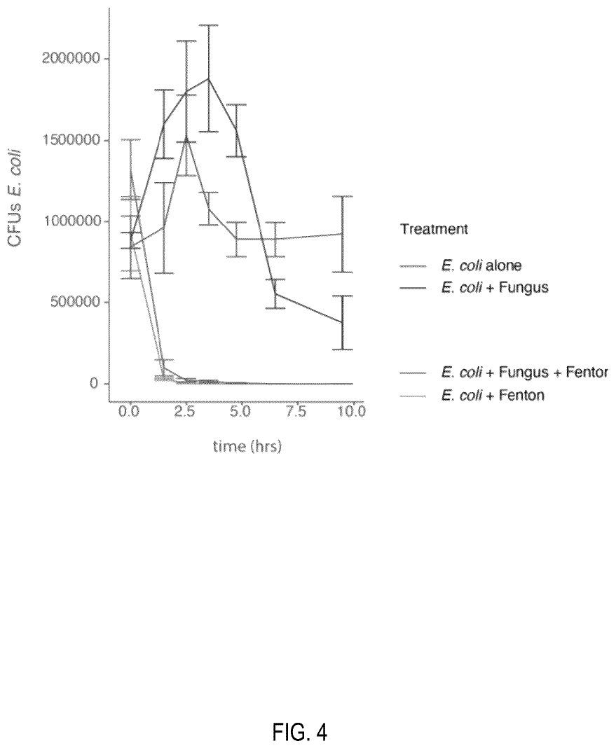

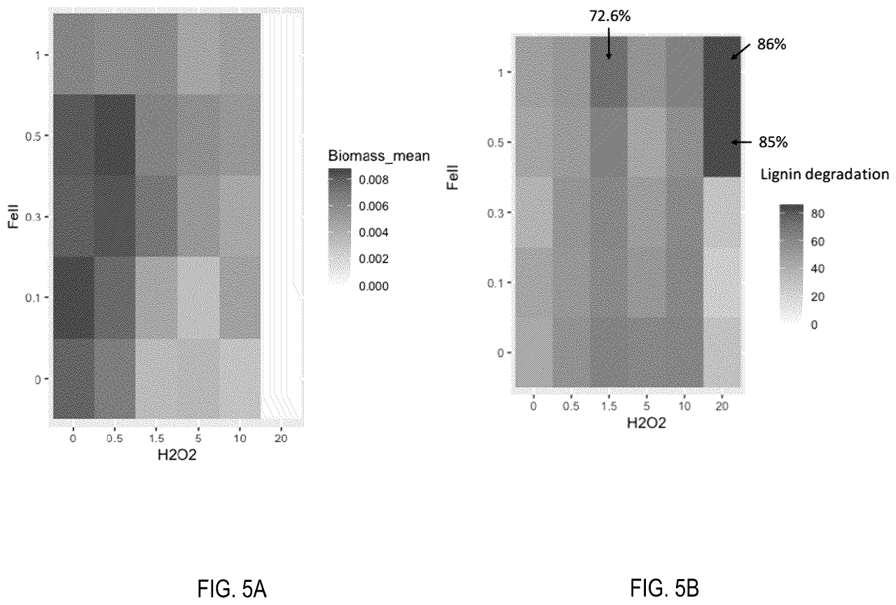

Referring now to FIGS. 5A-5B, fungal cultures consistent with embodiments of the present disclosure were investigated to demonstrate effective degradation of recalcitrant OCs. Specifically referring to FIG. 5A, fungal growth was found to be tolerant of wide ranges of Fenton reactant concentrations. Hydrogen peroxide concentrations below 0.5 mM and Fc (II) ion concentrations appeared to have no effect on fungal growth. Further, fungal growth appeared possible at concentrations approaching 20 mM H₂O₂. Referring specifically to FIG. 5B, enhanced lignin degradation was observed through the addition of Fenton reactants to a fungal culture including white rot fungi. In particular, concentrations of 1.5 mM H₂O₂ and 1 mM Fe(II) demonstrated lignin degradation capabilities approaching those of Fenton reactant-only reaction mediums including more than an order of magnitude greater H₂O₂ concentrations.

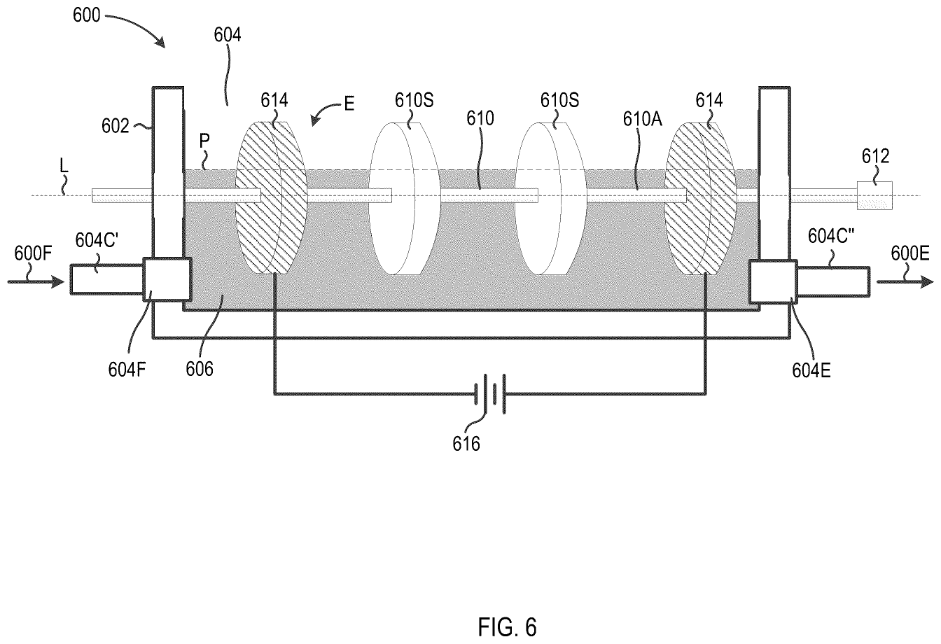

Referring now to FIG. 6, some embodiments of the present disclosure are directed to a system 600 for degrading OCs, e.g., lignin, perfluorochemicals, plastics, or combinations thereof. In some embodiments, system 600 includes a bioreactor 602. In some embodiments, bioreactor 602 includes one or more reservoirs 604. In some embodiments, a fungal culture 606 is included in reservoirs 604. Reservoirs 604 can be any suitable size and shape to hold fungal culture 606 and facilitate incubation and/or maintenance thereof.

In some embodiments, fungal culture 606 is consistent with the fungal cultures discussed above, working to degrade OC targets including recalcitrant OCs. In some embodiments, fungal culture 606 includes a plurality of OC-degrading fungi. In some embodiments, fungal culture 606 includes two or more different kinds strains of OC-degrading fungi. In some embodiments, the composition of the OC-degrading fungi component of fungal culture 606 evolves, e.g., exhibits enhanced native fungal diversity over time. In some embodiments, the OC-degrading fungi includes white rot fungi. In some embodiments, the white rot fungi includes Phanerochaete chrysosporium. In some embodiments, fungal culture 606 includes Fenton reactants. In some embodiments, fungal culture 606 includes a concentration of Fe(II) ions. In some embodiments, the Fe(II) ions are generated via dissociation of one or more iron compounds in fungal culture 606. In some embodiments, the concentration of Fe(II) ions in fungal culture 606 is between about 0.1 mM and about 1 mM. In some embodiments, the concentration of Fe(II) ions in fungal culture 606 is about 1 mM. In some embodiments, fungal culture 606 includes a concentration of hydrogen peroxide. In some embodiments, the concentration of hydrogen peroxide is less than about 20 mM. In some embodiments, the concentration of hydrogen peroxide is between about 0.5 mM and about 5 mM. In some embodiments, the concentration of hydrogen peroxide is about 1.5 mM. In some embodiments, the concentration of Fe(II) ions is about 1 mM and the concentration of hydrogen peroxide is about 1.5 mM.

Still referring to FIG. 6, in some embodiments, system 600 includes a feedstream 600F. In some embodiments, feedstream 600F is in fluid communication with reservoir 604, e.g., via an inlet 604F, conduit 604C′, etc., or combinations thereof. In some embodiments, feedstream 600F includes a concentration of one or more organic compounds. As discussed above, the OCs can be from any suitable source, e.g., industrial effluents, synthetic waste materials, naturally-occurring materials, etc., or combinations thereof.

In some embodiments, system 600 includes one or more effluent streams 600E in fluid communication with reservoir 604, e.g., via an outlet 604E, conduit 604C″, etc., or combinations thereof. In some embodiments, bioreactor 602 is operated as a batch reactor. In some embodiments, bioreactor 602 is operated as a semi-batch reactor. In some embodiments, bioreactor 602 is operated continuously.

In some embodiments, an incubation apparatus 610 is positioned within bioreactor 602. Incubation apparatus 610 is configured to aid proliferation of OC-degrading fungi in fungal culture 606 by providing one or more substrates upon which the fungi can attach and grow. In some embodiments, incubation apparatus 610 includes one or more shafts 610A. In some embodiments, shaft 610A extends longitudinally along reservoir 602. In some embodiments, one or more substrates 610S are positioned on shaft 610A and extend therefrom. Shaft 610A is configured to rotate on a longitudinal axis L, and in doing so cause substrates 610S to rotate within reservoir 602. In some embodiments, incubation apparatus 610 is submerged a predetermined percentage P into fungal culture 606. In some embodiments, as substrate 610S rotates within reservoir 604, e.g., via a motor 612, portions of the substrate are immersed in fungal culture 606 while other portions of the substrate are removed from the fungal culture. Thus, fungal growths attached to substrates 610S can be intermittently exposed to fungal culture 606 as well as an ambient environment E of reservoir 602, e.g., rich in oxygen gas. The amount of time that fungal growths attached to substrate 610S spend in fungal culture 606 can be controlled a function of at least the rotation speed of shaft 610A, the submergence percentage P, the size and shape of substrates 610S, or combinations thereof. In some embodiments, substrates 610S have a shape that includes a disk, rod, flange, etc., or combinations thereof. In some embodiments, substrate 610S extends obliquely from shaft 610A. In some embodiments, substrate 610S extends orthogonally from shaft 610A.

In some embodiments, bioreactor 602 includes one or more pairs of electrodes 614 in electrical communication with fungal culture 606 and a power supply 616. Electrodes 614 enable an electro-Fenton process, where additional hydrogen peroxide can be generated in reservoir 604 and further reduce Fenton reactant input burden on fungal culture 606. In some embodiments, electrodes 614 extend from shaft 610A, and further rotate therewith. In some embodiments, at least one electrode 614 is in electrical communication with fungal culture 606 but is not connected to incubation apparatus 610 (not pictured). In some embodiments, electrodes 614 are disks positioned on shaft 610A, e.g., as with exemplary embodiments of substrate 610S described above. Electrodes 614 can have any suitable composition for use with fungal culture 606, e.g., Fe₂O₃-graphite, carbon fiber, etc., or combinations thereof.

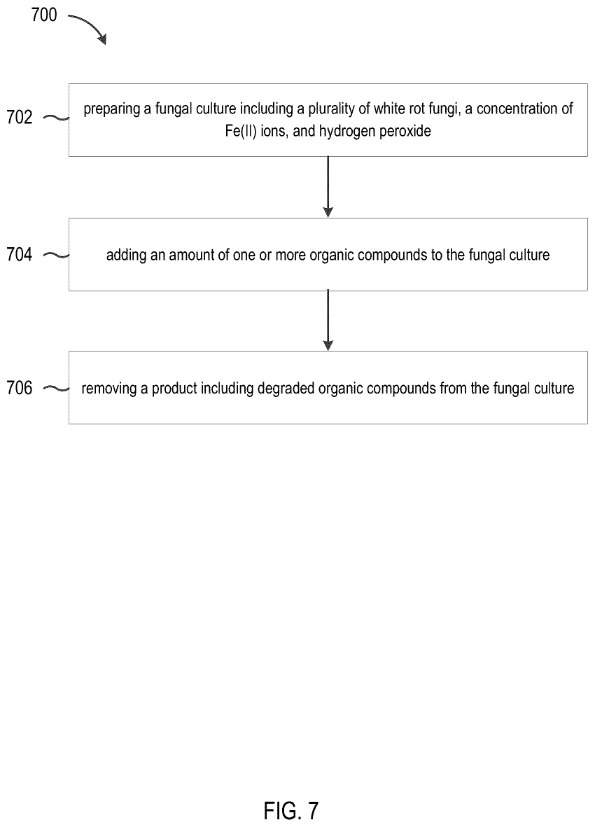

Referring now to FIG. 7, some embodiments of the present disclosure are directed to a method 700 of degrading organic compounds. In some embodiments, at 702, a fungal culture is prepared. As discussed above, in some embodiments, the fungal culture is configured to degrade OC targets including recalcitrant OCs. In some embodiments, the fungal culture includes a plurality of OC-degrading fungi. In some embodiments, the fungal culture includes two or more different kinds strains of OC-degrading fungi. In some embodiments, the composition of the OC-degrading fungi component of the fungal culture, e.g., exhibits enhanced native fungal diversity over time. In some embodiments, the OC-degrading fungi includes white rot fungi. In some embodiments, the white rot fungi includes Phanerochaete chrysosporium. In some embodiments, the fungal culture includes Fenton reactants. In some embodiments, the fungal culture includes a concentration of Fe(II) ions. In some embodiments, the Fe(II) ions are generated via dissociation of one or more iron compounds in the fungal culture. In some embodiments, the concentration of Fe(II) ions in the fungal culture is between about 0.1 mM and about 1 mM. In some embodiments, the concentration of Fe(II) ions in the fungal culture is about 1 mM. In some embodiments, the fungal culture includes a concentration of hydrogen peroxide. In some embodiments, the concentration of hydrogen peroxide is less than about 20 mM. In some embodiments, the concentration of hydrogen peroxide is between about 0.5 mM and about 5 mM. In some embodiments, the concentration of hydrogen peroxide is about 1.5 mM. In some embodiments, the concentration of Fe(II) ions is about 1 mM and the concentration of hydrogen peroxide is about 1.5 mM.

Still referring to FIG. 7, at 704, an amount of one or more OCs is added to the fungal culture. The fungal culture then works to degrade the OCs. As discussed above, in some embodiments, the OCs include lignin, perfluorochemicals, plastics, or combinations thereof. In some embodiments, at 706, a product including OCs degraded from the fungal culture is removed from the fungal culture, e.g., for postprocessing, disposal, etc., or combinations thereof.

Systems and methods of the present disclosure advantageously utilize the activity against organic compounds of white rot fungi in combination with Fenton reactants to degrade recalcitrant OCs, e.g., lignins, perfluorochemicals including PFOA, plastics and related compounds, or combinations thereof. Fungal cultures including white-rot fungi, 1.5 mM H₂O₂, and about 1 mM Fe²⁺ exhibited lignin degrading capabilities comparable to cultures of Fenton-only reaction mediums with significantly higher concentrations of hydrogen peroxide. Further, the combination of white rot fungus, e.g., P. Chrysosporium, and Fenton reactants act synergistically. Specifically, the Fenton reaction imposes oxidative stress on faster-growing microbial competitors to the white rot fungi like E. coli, selectively inhibiting the competing bacteria and their effect on fungal activity. The white rot fungi recycle Fe(II) ions, reducing the Fenton reactant input burden on the fungal culture. Incorporating an electro-Fenton reaction into the systems and methods of the present disclosure further reduces this burden by replenishing H₂O₂. The resulting systems and methods demonstrate OC degradation capabilities greater than with white rot fungi cultures or Fenton chemistry alone, resulting in more economical and sustainable treatment of recalcitrant OCs.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A fungal culture, comprising:
a plurality of organic compound (OC)-degrading fungi;
a concentration of Fe(II) ions; and
a concentration of hydrogen peroxide.

2. The fungal culture according to claim 1, wherein the OC-degrading fungi includes white rot fungi.

3. The fungal culture according to claim 2, wherein the white rot fungi includes Phanerochaete chrysosporium.

4. The fungal culture according to claim 1, wherein the concentration of Fe(II) ions is between about 0.1 mM and about 1 mM.

5. The fungal culture according to claim 1, wherein the concentration of hydrogen peroxide is less than about 20 mM.

6. The fungal culture according to claim 5, wherein the concentration of hydrogen peroxide is between about 0.5 mM and about 5 mM.

7. The fungal culture according to claim 6, wherein the concentration of hydrogen peroxide is about 1.5 mM.

8. The fungal culture according to claim 7, wherein the concentration of Fe(II) ions is about 1 mM.

9. The fungal culture according to claim 1, further comprising a concentration of organic compounds including wherein the organic compounds include lignin, perfluorochemicals, plastics, or combinations thereof.

10. A system for degrading organic compounds, comprising:
a bioreactor including one or more reservoirs;
a fungal culture in the one or more reservoirs; and
a feedstream in fluid communication with the reservoir, the feedstream including a concentration of one or more organic compounds,
wherein the fungal culture includes a plurality of white rot fungi, a concentration of Fe(II) ions, and a concentration of hydrogen peroxide.

11. The system according to claim 10, further comprising one or more pairs of electrodes in electrical communication with the fungal culture.

12. The system according to claim 10, further comprising an incubation apparatus positioned within the bioreactor, the incubation apparatus including:
a shaft extending longitudinally along the reservoir;
one or more substrates positioned on and extending from the shaft; and
a motor configured to rotate the shaft.

13. The system according to claim 12, wherein the substrate is a disk extending at least substantially orthogonally from the shaft.

14. The system according to claim 10, wherein the organic compounds include lignin, perfluorochemicals, plastics, or combinations thereof.

15. The system according to claim 10, wherein the white rot fungi includes Phanerochaete chrysosporium.

16. The system according to claim 10, wherein the concentration of hydrogen peroxide is between about 0.5 mM and about 5 mM.

17. The system according to claim 16, wherein the concentration of hydrogen peroxide is about 1.5 mM and the concentration of Fe(II) ions is about 1 mM.

18. A method of degrading organic compounds, comprising:
preparing a fungal culture including a plurality of white rot fungi, a concentration of Fe(II) ions, and hydrogen peroxide; and
adding an amount of one or more organic compounds to the fungal culture,
wherein the concentration of Fe(II) ions in the composition is about 1 mM and the concentration of hydrogen peroxide in the composition is about 1.5 mM.

19. The method according to claim 18, wherein the white rot fungi includes Phanerochaete chrysosporium.

20. The method according to claim 18, wherein the organic compounds include lignin, perfluorochemicals, plastics, or combinations thereof.
